(19) United States
(12) Patent Application Publication
(10) Pub. No.: US 2013/0042522 A1
(43) Pub. Date: Feb. 21, 2013

(54) Ponds for the culture of photosynthetic organisms

(71) Applicant: Dominique Delobel, Carvin (FR)

(72) Inventor: Dominique Delobel, Carvin (FR)

(73) Assignee: ALGAESTREAM, Paris (FR)

(21) Appl. No.: 13/643,807

(22) PCT Filed: Apr. 28, 2011
(86) PCT No.: PCT/FR2011/000259

§ 371 Date: Oct. 26, 2012

(30) Foreign Application Priority Data
Apr. 29, 2010 (FR) 1001832

(51) Int. Cl. A01H 13/00; E02B 1/00

(52) U.S. Cl. 47/1.4; 405/270

(57) ABSTRACT

The present invention relates to a method for the construction of land-based covered ponds for the aquaculture of photosynthetic organisms and the associated device constituted by a very long gutter which serves both as a wall shared by two juxtaposed ponds, support for the covering of the ponds, passageway for the operating staff and rainwater receptacle.

The invention is particularly suitable for equipping large farms dedicated to the production of algae or other photosynthetic organisms.

The present invention describes a covered pond, advantageously (but not exclusively) intended for equipping farms for the land-based aquaculture of photosynthetic organisms and the technical device necessary for its realization.

The field of the invention therefore relates to covered ponds of all sizes, particularly those with a unit size equal to or greater than 500 square metres.

A farm for the land-based aquaculture of photosynthetic organisms is constituted by a set of ponds often identical in shape but in several series of sizes. For the sake of efficiency the ponds are arranged so as to optimize land use, which most often results in an alignment of identical ponds in one or more rows.

Each pond is separated from its immediate neighbour by a space which allows the free movement of the operating staff and equipment and which can serve to support the networks of the installation such as channels, pipes, cables.

The ponds are shallow in order to ensure the maximum diffusion of sunlight throughout the liquid medium and are most often constructed or positioned above ground.

The walls of said ponds are generally constituted by concrete barriers, solid or hollow bricks, boards, an earth or compacted sand embankment, or any other suitable material or device.

The low height of the contained liquid, most often limited to a few tens of centimetres, leads to a low lateral pressure on the walls, such as for example less than 5000 Pascals for a height of liquid of 50 centimetres, which allows the use of thin or flexible walls utilized according to accepted practice.

The covering of the ponds, irrespective of the particular embodiment is

a) individual: each pond has its own covering which is supported either on the pond wall itself which in this case must be reinforced and suited to supporting this load or, most often, outside in order to include a passageway around the whole perimeter of the pond, inside the covering. The span of the structure is at least equal to the width of the pond optionally increased by the width of the lateral passageways.

b) collective: the same covering takes in several ponds. The ground anchors of the support structure of this covering are spaced out regularly according to accepted practice and positioned in the spaces between the ponds where they constitute obstacles to movement. The span of the structure is at least equal to the width of a single pond increased by the width of a passageway between two ponds.

These coverings often take the form of greenhouse tunnels or multispan greenhouses known to a person skilled in the art and the height of which is a few metres, generally between 3 and 5, which results in a significant wind surface area.

The functions ensured by the coverings include that of dealing with precipitation, in particular rain. The rainwater is systematically guided to the periphery of the covering, at one or more points depending on the shape of the support structure of said covering, and depending on the forms and fittings suited to each particular embodiment.

The rainwater is conveyed to the ground into the space normally intended for movement between the (individual or collective) coverings and must be dealt with by any appropriate means such as, by way of example, a channel, pipe, culvert, in order to be removed outside the pond zone, using the equipment or arrangements known to a person skilled in the art and depending on the requirements due to the site topography such as ditches, sumps, lifting pumps, retention pond.

Rainfall over a surface area of one hectare of covered ponds represents a volume of one hundred cubic metres per ten millimetres of precipitation. Such quantities must be removed in order to avoid damage such as the flooding of the installations, displacement or destruction of the walls of the ponds, gully erosion or destabilization of the ground.

The invention overcomes some of these drawbacks and helps to facilitate the deployment of the ponds and reduce the costs thereof by the economical use of materials at least.

The invention is based on the laying on the ground of a set of very long gutters, advantageously regularly spaced out, so as to allow the installation of the ponds between the gutters, said gutters being able to act simultaneously as pond wall, support for the covering of said ponds, passageway for the operating staff, support for the networks, and collection and conveyance of rainwater out of the pond area.

Thus a first subject of the invention is a device for the construction of walls of covered ponds, comprising a very long profiled gutter\textcircled{4}, advantageously laid on the ground, which can act as a wall, advantageously a wall for two juxtaposed ponds, and the profile of which can allow the pond liner films\textcircled{9} and covering films\textcircled{10} to be fixed over the entire length of the gutter.

A subject of the invention is also a covered pond, characterized by the utilization of at least one very long profiled gutter\textcircled{4} as sidewall, advantageously shared by two juxtaposed ponds, and as support for the flexible air- and water-tight top covering of each pond combined with the use of flexible films or sheets as pond liners.

The attached drawings illustrate the invention.

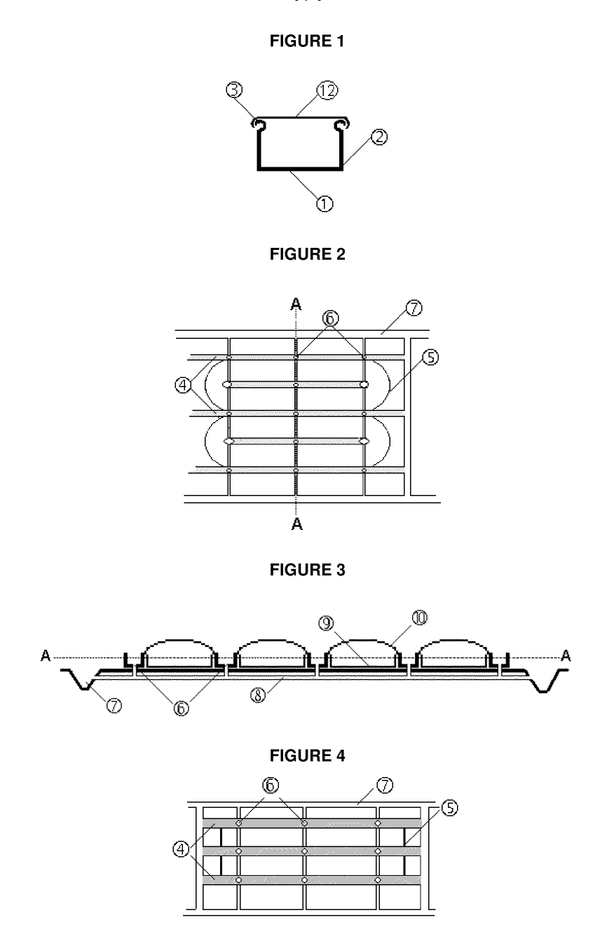

FIG. 1 represents a cross-sectional view of the profile of a particular embodiment of the gutter.

FIG. 2 represents a top view of a set of ponds produced using the gutter.

FIG. 3 represents a cross-section along AA in FIG. 2.

FIG. 4 represents a top view of a set of ponds produced using the gutter (variant of FIG. 2).

The gutter\textcircled{4}, an essential device of the invention, can take the usual form known to a person skilled in the art in its conventional use.

In the particular use specific to the invention, said gutter\textcircled{4}, can be very long, up to several hundred metres, advantageously from 5 to 500 metres, even more advantageously from 10 to 300 metres. Said gutter\textcircled{4} can have a substantially U-shaped cross-sectional profile with a base and sidewalls, advantageously like that described by FIG. 1. The base \textcircled{1}, optionally laid directly on the ground can be from a few tens of centimetres to several metres wide, for example from 10 centimetres to 10 metres, advantageously from 30 centimetres to 3 metres, even more advantageously from 40 to 100 centimetres. The sidewalls\textcircled{2}, advantageously vertical, can be from 5 to 100 centimetres, advantageously from 10 to 30 centimetres high and have at their top edge a fold \textcircled{3} which can run the entire length of the gutter.

The longitudinal fold \textcircled{3} can be intended to i) ensure the conventional function of stiffening the wall \textcircled{2} in order to limit deformation thereof, ii) allow the attachment of the flexible pond liner and covering films (see below).

According to the particular embodiment, this fold can be rounded in shape or comprise sharp edges, and be oriented towards the outside or towards the inside of the gutter.

According to a particular embodiment, not shown, said fold \textcircled{3} can be duplicated. A first fold can be positioned at the top of the sidewall \textcircled{2} and can ensure the stiffening thereof. A second fold can be positioned between the top and the base of the sidewall \textcircled{2} and can ensure the function of attachment of the flexible films (see below).

Said gutter necessary for carrying out the invention can be manufactured by any appropriate technical means known to a person skilled in the art such as by way of non-limitative examples:

i) cold profiling of a very long sheet (several tens or hundreds of metres), advantageously presented as a roll, of a deformable thin metal such as aluminium, galvanized or stainless steel, or any other metal which may be suitable because of its technical characteristics or its cost (technique of producing metal gutters by means of a cold profiling machine).

ii) profile extrusion of a thermoplastic material in sections of a few metres assembled by adhesive bonding, welding, bolting or riveting in order to obtain the required great length (technique for producing PVC tubes or pipes).

iii) moulding of pieces of concrete or cement joined end to end in order to obtain the required great length (technique for producing culverts or kerbs).

According to the general embodiment of the invention said gutters can be simply laid on ground which has optionally been levelled and, if necessary, compacted beforehand according to the appropriate techniques known to a person skilled in the art.

These civil engineering operations can be carried out so as to:

i) produce a support surface covering the entire surface area occupied by a set of n interdependent ponds

ii) produce an almost perfectly flat surface so that any two points on the entire support surface defined in (i) have a maximum difference in height less than or equal to 5 centimetres and ideally less than or equal to 2 centimetres, a level of tolerance largely within the scope of specialist enterprises for example in the production of road surfaces.

iii) obtain a surface layer free of any element capable of attacking the flexible materials which may be deployed there in order to constitute the pond liners\textcircled{9}. The putting in place of an adjusting layer of fine sand after stone removal is an example of a solution for achieving this objective.

The gutters \textcircled{4} can be arranged on the support surface thus produced, in accordance with the following conditions:

i) all the gutters in the same set can ideally be produced identically, irrespective of the particular method of production and can at least have a single cross-sectional profile (same design, same dimensions).

ii) the gutters can be laid strictly parallel.

iii) the distance between the gutters can determine the width of the ponds.

iiii) the gutters can be substantially identical in length. This length can determine that of the ponds, which is slightly less as a general rule.

iiiii) the gutters can be firmly held in place by any means appropriate to the particular embodiment such as for example 1) by the inherent weight of the gutter produced from concrete, 2) by partial filling with any appropriate material such as sand, water, gravel, concrete or other, 3) by spikes fixed in the ground by any appropriate means through the base \textcircled{1} or tangentially to the walls\textcircled{2}.

The base of the ponds can be produced using a water-tight flexible film \textcircled{9} the width of which is equal to the distance between two gutters increased by double the height of the vertical wall \textcircled{2} of said gutter. The length of said flexible film must be equal to that of the gutters. According to the invention, said flexible film \textcircled{9} can be made of any material compatible with its intended function, such as for example, and without limitation, ethylene-propylene-diene monomer (EPDM) membranes, vinyl polychloride or polyvinyl chloride (PVC) sheet, polyethylene (PE) film.

Accepted practice for laying the pond liner film can require the laying of a protective layer of a suitable material, such as by way of example a geotextile felt, beforehand.

According to the particular embodiment, the chosen materials, their technical characteristics and the applicable state of the art, the pond liner can be constituted by the superimposition of several flexible films.

The covering of the pond \textcircled{10} can be ensured by an air- and water-tight film permeable to visible light. A polyethylene film commonly used in agriculture can correspond to these characteristics.

The width of the covering film can be substantially equal to that of the pond liner film.

Each of the two films, pond liner and covering, can be unrolled and arranged tensioned and without folds on the ground and over the entire surface area of the pond.

The longitudinal edges of the two superimposed films can be brought together against the upper rim on the same side of the lateral gutter in order to be fixed there over the entire length of the gutter in continuous manner, by any means suited to the particular embodiment.

By way of example, this fixing can be achieved by a rubber flange compressing the two films inside the fold\textcircled{3}, or by any suitable system or device capable of ensuring:

i) the holding in position of the liner film which must withstand the pressure and the movement of the liquid contained in the pond.

ii) the holding in the high position of the covering film \textcircled{10} which must withstand the effects of the wind and application of positive pressure to the air contained in the space included between the surface of the liquid contained in the pond and said covering film.

iii) the holding in the low position of the covering film when the latter is covered with a layer of water, intentionally deposited within the context of the exploitation of the ponds or as an unintentional but foreseeable result of an excess of rain.

The production of the ends\textcircled{5} of the ponds remains outside the scope of the invention. According to each particular embodiment, illustrated by FIGS. 2 and 4, these ends can take different shapes while still complying with two common rules:

i) the walls of the ends must ensure the holding in the high position of the liner film so as to ensure the tightness of the pond and the necessary retention of the liquid contained.

ii) the continuity of the bond between the flexible liner and covering films must be ensured so as to maintain the containment of the liquid and gaseous content of the pond by incorporating, if appropriate, rigid technical devices made necessary by any particular embodiment of said ponds.

For each pond constructed according to the invention:
the gutter can represent the only rigid structure over the entire length of the pond,
there may be no transverse rigid structure,
the holding of the covering in the high position can be ensured by applying a positive pressure to the air or the gas or gases contained in said pond. This positive pressure can be achieved by any suitable device known to a person skilled in the art such as a fan, a compressor or a blower.

The pond covering can be mobile and variable in height, and can, depending on the needs of the operation, be maintained in the normal high position or in the low position in contact with the liquid contained in the pond by simply stopping the devices applying positive pressure.

This arrangement allows the security of the pond to be ensured i) by limiting the pond's wind surface area as far as possible ii) by submersion of the covering: the height of the liquid in the pond is necessarily lower than that of the walls \textcircled{2} of the gutters. A sufficient quantity of water to cover the entire pond surface area to a depth of a few centimetres can rapidly be made to spread over the (air- and water-tight) covering film. This operation makes it possible to present the set of ponds as an area of liquid and thus to withstand very violent winds capable of damaging or destroying the installations and in particular the pond coverings should they be held in the high position.

Applying a positive pressure to the inside of the pond can allow the normal operation of the installation to be resumed: the inflation and gradual raising of the covering displaces the top layer of water which flows towards the lateral gutters and can be removed according to the normal procedure.

According to a particular embodiment of the invention, in order to reinforce the wind resistance of the assembly, stiffeners\textcircled{12} can be arranged spaced out over the entire length of the gutters. The stiffener has two functions: i) to strictly maintain the distance between the walls\textcircled{2} of the gutter at the width of the base\textcircled{1}, ii) to compress the rim \textcircled{3} of the gutter in order to control the opening thereof and secure the fixing of the films \textcircled{9} and \textcircled{10}.

According to a general embodiment of the invention, with respect to the function of collecting rainwater and the great length of the ponds, the removal of the waters via the ends of the gutters only can prove insufficient as a function of the cross-section of the gutter.

In order to remedy this, it can be useful to have intermediate outlets \textcircled{6}, advantageously spaced out regularly along the length of the gutters relative to the rainfall in the particular geographical area of the installation (for example every fifty metres).

Said outlets can be connected to an underground pipeline \textcircled{8}, advantageously arranged perpendicular to the gutters. Each underground pipeline can be produced from materials and in suitable dimensions according to accepted practice, and can be installed before the gutters \textcircled{4} and the pond liner films are laid \textcircled{9} and can open, on either side of the pond area that it serves, into a ditch \textcircled{7} or main pipeline which has the function of conveying the collected water away from the pond zone.

The advantages of the invention for the construction of ponds with large dimensions reside i) in the economical use of resources: a single gutter serves as wall for two contiguous ponds, as support structure for the covering of said ponds, and as means for collecting and removing rainwater, ii) in the rapidity of installation of the ponds, iii) in the optimization of the occupation of the site by drastically reducing the space between the ponds, iiii) in reduced wind surface area by limiting the height of the covering to less than a quarter of the width of the pond.

The invention is particularly intended for equipping large farms for the culture of microalgae and photosynthetic organisms, in particular such as may result from the implementation of the invention which is the subject of the French patent no. 2 918 387.

A subject of the invention is also a device for the construction of walls of covered ponds according to the invention, comprising a very long profiled gutter laid on the ground, which acts as wall for two juxtaposed ponds juxtaposes, and the profile of which allows the pond liner and covering films to be fixed over the entire length of the gutter.

A subject of the invention is also the use of ponds according to the invention in aquaculture farms as well as said aquaculture farms comprising at least one pond according to the invention.

1. Device for the construction of walls of covered ponds comprising a very long profiled gutter\textcircled{4} advantageously laid on the ground, which acts as a wall, advantageously as a wall for two juxtaposed ponds, and the profile of which allows the pond liner\textcircled{9} and covering films\textcircled{10} to be fixed over the entire length of the gutter.

2. Device according to claim 1, characterized by the positioning of transverse stiffeners\textcircled{12} regularly spaced out over the entire length of said profiled gutter\textcircled{4}.

3. Covered pond, characterized by the utilization of at least one device according to any one of claim 1 or 2, comprising a very long gutter as sidewall, advantageously shared by two juxtaposed ponds, and as support for the flexible air- and water-tight top covering of each pond combined with the use of flexible films or sheets as pond liners.)

4. Pond according to claim 3, characterized in that it comprises a pond covering without a rigid transverse bearing structure, variable in height, directly supported by the walls of the gutter.

5. Pond according to any one of claim 3 or 4, characterized in that it comprises a device for securing the installations by submersion of the flexible and tight pond covering.

6. Pond according to any one of claims 3 to 5, characterized in that outlet devices intended for the flow of rainwater are positioned spaced out over the length of a gutter.

7. Use of a device according to any one of claim 1 or 2 in the construction of ponds.

8. Use according to claim 7 in an aquaculture farm, particularly a farm intended for the culture of photosynthetic organisms, very particularly a farm for the aquaculture of microalgae.
